(19) United States
(12) Patent Application Publication
(10) Pub. No.: US 2011/0039343 A1
(43) Pub. Date: Feb. 17, 2011

(54) METHOD FOR THE IDENTIFICATION OF PATIENTS IN NEED OF THERAPY HAVING MINOR COGNITIVE DISORDERS AND THE TREATMENT OF SUCH PATIENTS

(75) Inventor: Andreas Bergmann, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Brahms Aktiengesellschaft, Hennigsdorf (DE)

(21) Appl. No.: 12/865,492

(22) PCT Filed: Jan. 29, 2009
(86) PCT No.: PCT/EP09/51035

§ 371 Date: Oct. 25, 2010

(30) Foreign Application Priority Data
Feb. 1, 2008 (DE) 10 2008 007 218.4
Jul. 27, 2008 (EP) 08161228.5

(51) Int. Cl. G01N 33/68

(52) U.S. Cl. 436/86

(57) ABSTRACT

A method for the identification of patients in need of therapy and for the preventive treatment of such patients having minor cognitive disorders, wherein an increased risk can be determined for said patients to develop clinically manifested Alzheimer's disease during a preceding risk stratification as a result of an increase in circulation-relevant peptide biomarkers measurable in the circulation of said patients, using drugs that comprise one or more active ingredients of cardiovascular agents, which are selected from the group consisting of ACE inhibitors, angiotensin II receptor antagonists, betablockers and blood pressure-lowering diuretics and the combinations thereof, or using drugs comprising one or more ANP receptor antagonists or one or more adenosine receptor antagonists.

The present invention relates to a new in vitro method of risk stratification of patients with mild cognitive disorders for the purpose of identifying patients requiring treatment as they are at increased risk of deterioration of their originally mild cognitive disorders and of the development of a clinically manifest neurodegenerative disease of Alzheimer's dementia type, as well as for the purpose of providing such patients requiring treatment with certain drugs, the suitability of which for treatment purposes in this connection constitutes knowledge which forms an essential part of this invention.

The invention also relates to a method for the preventive treatment of certain risk patients with mild cognitive disorders, identified on the basis of a biomarker measurement, with cardiovascular agents in order to prevent or delay the development of clinical manifest neurodegenerative disorders of Alzheimer's dementia type, or the use of the active substances of cardiovascular agents and the use of ANP receptor antagonists in such a method as well as for the production of drugs for said purpose of preventing or delaying the development of clinically manifest neurodegenerative Alzheimer's dementia-type disorders.

The patients for whom, within the framework of the present application, a retrospective assessment of their initial risk of subsequently developing a confirmed clinically manifest neurodegenerative disease was carried out and in whom in addition, factors were determined from their previous medical histories (medical files) which retrospectively proved to be prognosis-improving, are patients diagnosed with “mild cognitive disorders” (abbreviated to MCD). In the English-language literature the criteria for confirming “mild cognitive disorders” have more recently been handled under the term “mild cognitive impairment” (abbreviated to MCI). With regard to this reference is made for example to Serge Gauthier et al., “Mild cognitive impairment” in: Lancet 2006, 367:1262-1270, as well as to Ronald C. Petersen et al., “Mild Cognitive Impairment, Clinical Characterization and Outcome”, In: Arch Neurol. 1999, 56:303-308, or Ronald C. Petersen et al., “Current Concepts in Mild Cognitive Impairment” in: Arch Neurol. 2001; 58:1985-1992. One of the test methods used in connection with determining a state of MCD or MCI in a patient examines a parameter known as the “Mini Mental State” (MMS) of a person being assessed, cf. Marshal F. Folstein et al., “MINI-MENTAL STATE—a practical method for grading the cognitive state of patients for the clinician”, in: J. Psychiat. Res., 1975, 189-198.

It is known that only in some of those patients in whom at a certain point in time, on the basis of own observations or observations of their environment, “mild cognitive disorders” (MCD) have been determined, or who fulfil the criteria for “mild cognitive impairment” (MCI), will a progressive deterioration of their condition be observed. Certain patients only gradually develop the full clinical symptoms of a neurodegenerative disease, e.g. of Alzheimer's dementia (AD) type. In others a different type of dementia, e.g. vascular dementia, develops. In yet other MCD or MCI patients no deterioration of any note develops and sometime the original symptoms even disappear. The first group, in whom a neurodegenerative disease develops is also known as the “converters to Alzheimer'” (converters) group, whereas the latter group is the “non-converters” group.

Given the described initial situation, it would be desirable within the group of patients diagnosed with “mild cognitive disorders” to be able to assess at the time of diagnosis the individual risk of whether the patients are likely to belong to the group of “converters to Alzheimer's” or not, so that if a high risk is determined early preventive or therapeutic measures can be initiated (if such measures exist) while determining only a low risk of such conversion could avoid unnecessary measures and relieve the patients of a considerable psychological burden.

It would also be desirable to be able to provide patients who are at increased risk of conversion to Alzheimer's with a treatment, the long-term efficacy of which is guaranteed in the sense of avoiding or delaying a conversion.

The aim of the present invention is to provide a method that allows identification of the group of converters to Alzheimer's with a high statistical probability, and that also creates a basis for deciding whether to provide the identified converters with effective preventive treatment.

These objectives are achieved by methods and their preferred embodiments which can be gleaned from the claims by a person skilled in the art.

One of the aspects of the invention embodies an analytical in vitro method, which in a group of patients diagnosed with “mild cognitive disorders” on the basis of clinical findings, allows the identification at the time of diagnosis of patients at high risk of subsequent conversion to Alzheimer's, and also allows a specific treatment recommendation to be made for these patients.

Within the framework of the present invention, as a first stage an analytical in vitro method is used to determine the concentration of circulation-relevant peptide biomarkers in patient samples (plasma, serum), and in the event of determining concentrations found to be elevated compared with corresponding reference concentrations, the patients in question are recommended treatment with cardiovascular agents, more particularly antihypertensive agents or treatment with ANP receptor antagonists, as the evaluations described below have shown that treatment with antihypertensive agents or with ANP receptor antagonists reduces the risk of an identified mild cognitive disorder deteriorating into a manifest neurodegenerative disease of Alzheimer's dementia (AD) type.

The subject matter of the present invention is therefore an in vitro method of identifying patients with mild cognitive disorders requiring treatment, in which:
at least one circulation-relevant peptide biomarker is determined in a sample from a biological fluid from the circulation of a patient diagnosed with a mild cognitive disorder, and
an increased risk of developing a clinically manifest neurodegenerative disease is allocated to a concentration of the at least one circulation-relevant peptide biomarker exceeding a biomarker-specific threshold value (cut-off) set in the range of concentration values for this marker usual in the case of mild cognitive disorders concentration.

For patients with a determined higher risk a recommendation for treatment with medication in accordance with the following can be made.

In accordance with the in vitro method of the invention the sample is preferably a serum or plasma sample.

Preferably the circulation-relevant peptide biomarkers are selected from the natriuretic peptides ANP and/or BNP and/or adrenomedullin (ADM) or from fragments of their associated prohormones pro-ANP, pro-BNP or pro-ADM.

The method in accordance with the invention is preferably also characterised in that a treatment is recommended with a cardiovascular agent product from the group of antihypertensive agents selected from the group of inhibitors of “Angiotensin Converting Enzyme” (ACE inhibitors), angiotensin II receptor antagonists (“sartans”), beta blockers or diuretics used to lower blood pressure. Alternatively, treatment with an ANP receptor antagonist or an adenosine receptor antagonist can be recommended.

In a preferred embodiment the determination is carried out with an immuno-diagnostic determination method. It is also preferable to determine fragments of prohormone precursors of circulation-relevant peptides with a physiological effect which is reduced or undetectable with regard to the actual circulation-relevant peptides.

In a particularly preferred embodiment the method is characterised in that the concentration of a pro-ANP fragment (MR-pro-ANP) containing the middle section of the pro-ANP and/or of a mid-regional pro-ADM fragment (MR-proADM) containing the amino acids 45-92 of the pre-proadrenomedullin is determined.

Within the context of the invention preferred threshold values for MR-ProANP are 50-100 pmol/l, preferably 60-90 pmol/l and for MR-ProADM 0.4-0.8 nmol/l, preferably 0.5-0.7 nmol/l

The subject matter of the present invention is also the use of circulation-relevant peptide biomarkers, particularly natriuretic peptides ANP, BNP and/or adrenomedullin (ADM) or fragments thereof or fragments of their associated prohormones pro-ANP, pro-BNP or pro-ADM for the stratification or identification of patients with mild cognitive disorders at increased risk of conversion to a clinically manifest neurodegenerative disorder.

In accordance with another of its aspects the invention relates to a method of preventive treatment of patients with mild cognitive disorders, in whom a significant risk has been determined of deterioration of their condition into a clinical manifest neurodegenerative disorder of Alzheimer's type (AD risk patients), with drugs which contain one or more active substances of cardiovascular agents, more particularly antihypertensive agents, as well as drugs containing one or more ANP receptor antagonists.

In accordance with another of its aspects the present invention relates to the use of one or more active substances of cardiovascular agents as well as the use of one or more ANP receptor antagonists, more particularly antihypertensive agents, for the preventive treatment of AD risk patients or to the production of drugs for the preventive treatment of AD risk patients.

The subject matter of the invention is a drug, which contains one or more active substances of cardiovascular agents, selected from the group comprising: ACE inhibitors, angiotensin II receptor antagonists, beta blockers and antihypertensive diuretics and combinations thereof, for treating patients with mild cognitive disorders in order to delay or prevent the development of clinically manifest neurodegenerative disorders, or to improve or maintain the status of the mild cognitive disorder

Further, the subject matter of the present invention is a drug containing an antagonist of the atrial natriuretic peptide receptor (ANP receptor) to treat patients with mild cognitive disorders in order to delay or prevent the development of clinically manifest neurodegenerative disorders or to improve or maintain the status of the mild cognitive disorder.

Thus, the subject matter of the invention is the use of a drug containing an antagonist of the atrial natriuretic peptide receptor for producing a drug to treat patients with mild cognitive disorders in order to delay or prevent the development of clinically manifest neurodegenerative disorders or to improve or maintain the status of the mild cognitive disorder.

The subject matter of the invention are also therapeutic methods for treating patients with mild cognitive disorders in which a therapeutically effective quantity of ANP receptor antagonist is administered in order to delay or prevent the development of clinically manifest neurodegenerative disorders or to improve or maintain the status of the mild cognitive disorder.

Further, the subject matter of the present invention is a drug containing an adenosine receptor antagonist for treating patients with mild cognitive disorders in order to delay or prevent the development of clinically manifest neurodegenerative disorders or to improve or maintain the status of the mild cognitive disorder.

Thus, the subject matter of the invention is the use of a drug containing an adenosine receptor antagonist for producing a medicament for treating patients with mild cognitive disorders in order to delay or prevent the development of clinically manifest neurodegenerative disorders or to improve or maintain the status of the mild cognitive disorder.

The subject matter of the invention is thus also a drug containing one or more active substances of cardiovascular agents, selected from the group comprising: ANP receptor antagonists, adenosine receptor antagonists, ACE inhibitors, angiotensin II receptor antagonists, beta blockers and antihypertensive diuretics and combinations thereof to treat patients with mild cognitive disorders in order to delay or prevent the development of clinically manifest neurodegenerative disorders or to improve or maintain the status of the mild cognitive disorder.

The subject matter of the invention are also therapeutic methods for treating patients with mild cognitive disorders in which a therapeutically active quantity of adenosine receptor antagonists is administered in order to delay or prevent the development of clinically manifest neurodegenerative disorders or to improve or maintain the status of the mild cognitive disorder.

Adenosine receptors are a class of purinergic, G-protein-coupled receptors (GPCRs), the endogenous ligand of which is adenosine. Four types can be distinguished: A1, A2A, A2B and A3 the distribution in the body and function of which is partially overlapping and partially different. The adenosine receptor subtypes also differ from each other in terms of biochemical effects and signal pathways. Agonists and antagonists are known for each of these subtypes, with some agonists and antagonists being non-specific and showing effects with all adenosine receptors.

The subject matter of the invention are also therapeutic methods of treating patients with mild cognitive disorders in which a therapeutically active quantity of one or more active substances of cardiovascular agents is administered in order to delay or prevent the development of clinically manifest neurodegenerative disorders or to improve or maintain the status of the mild cognitive disorder.

Such cardiovascular agents are selected in particular from the group comprising: ACE inhibitors, angiotensin II receptor antagonists, beta blockers and antihypertensive diuretics and combinations thereof.

Antagonists of ANP receptors in accordance with the invention are therapeutically administrable substances that bind to ANP receptors, thereby preventing or reducing the binding of the natural ligand ANP, and do not lead to an activation of ganylate cyclase, resulting in no or reduced increase in cGMP and consequently a reduction in the natriuretic effect of the endogenous ANP.

Adenosine receptor antagonists in accordance with the invention are therapeutically administrable substances which bind to adenosine receptors, thereby preventing or reducing the binding of the natural ligand adenosine, and do not lead to an activation of the ganylate cyclase, resulting in no or reduced increase in cGMP and consequently a reduction in the effect of the endogenous adenosine.

In a particularly preferred embodiment of the above invention the ANP receptor antagonist is a glucose caproic acid polymer (glucose caproic acid, more particularly β1→6 glucane esterified with caproic acid). A particularly important representative of ANP receptor antagonists is HS-142-1 (U.S. Pat. No. 5,132,112). All antagonists mentioned in U.S. Pat. No. 5,132,112 form part of the present description and are ANP receptor antagonists in accordance with the invention. The HS-142-1-compounds are characterised as follows in U.S. Pat. No. 5,132,112:

These compounds comprise linear β1→6 glucane esterified with caproic acid whereby the number of D-glucose residues is 28 and the number of caproic acid residues is 11.

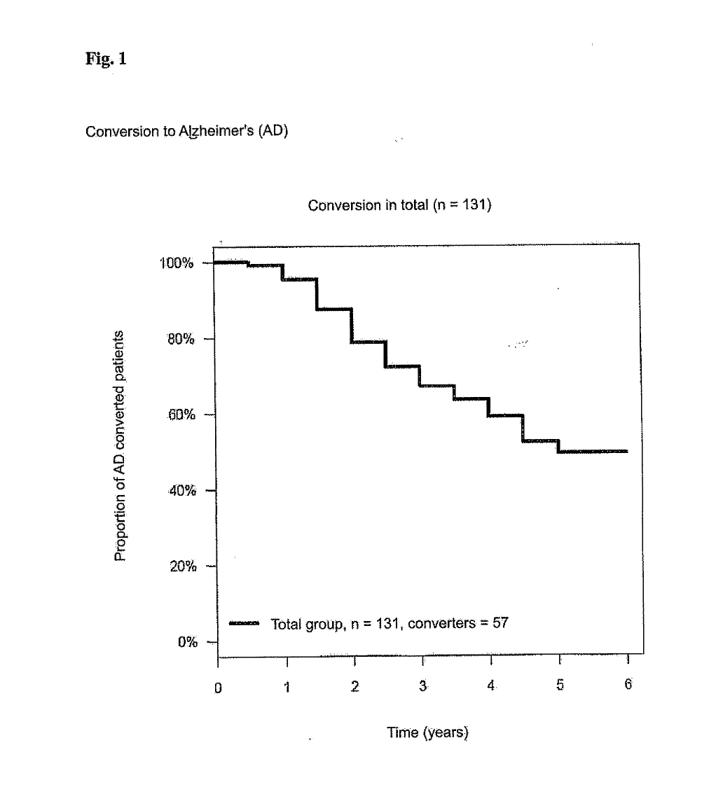

These compounds essentially have the following physicochemical properties:
1) Appearance: white powder
2) Melting point: 175-185° C.
3) Molecular formula: C₂₃₄H₃₉₂O₁₅₂
4) Mass spectrum (negative FAB mass spectrum):
   Found: M/Z 5536.5 (M-H)⁻
   Calculated: M/Z 5637.3
5) Specific rotation: [α]²⁴_{D}=−29.4° (c 0.24, aqueous solution)
6) Infra-red absorption spectrum (KBr tablet method):
   cm⁻¹: 3420, 2930, 1730, 1635, 1455, 1380, 1250, 1170, 1045
7) Ultra-violet absorption spectrum (aqueous solution): only terminal absorption visible.
8) ¹H-NMR spectrum (500 MHz, in D₂O): as shown in FIG. 1 of U.S. Pat. No. 5,132,112.
9) Colour reaction:
   Positive reactions with anisaldehyde, sulphuric acid and iodide.
   Negative reactions with ninhydrine, dinitrophenylhydrazine, iron chloride, bromocresol green and Dragendorff's reagent.

Further, ANP receptor antagonists are mentioned in WO 89/00428 and are also part of this description. It is also known that some ANP derivatives can exhibit antagonistic properties (JP-A-225399/88)—these antagonists too are part of this description.

Lipo-oligosaccharides, such as HS-142-1 are known as antagonists of the ANP receptor, such antagonists and their production are described in Yi Qiu, et al. Biosci. Biotech, Biochem, 60 (8)1308-1316, 1996 and are incorporated here as such as is their production. In particular these involve gentiohexaosyl derivatives as ANP receptor antagonists. Examples of such derivatives are: HS-142-1, O-(3-O-caproyl-β-D-glucopyranosyl)-(1→6)-[O-(β-D-glucopyranosyl)-(1→6)-O-(3-O-caproyl-β-D-glucopyranosyl)-(1→6)]₂-D-glucopyranose (1), O-(3-O-hexyl-β-D-glucopyranosyl)-(1→6)-[O-(β-D-glucopyranosyl)-(1→6)-O-(3-O-hexyl-β-D-glucopyranosyl)-(1→6)]₂-D-glucopyranose (4) and O-(3-O-caproyl-2,4,6-tri-O-methyl-β-D-glucopyranosyl)-(1→6)]₂-2,3,4-tri-O-methyl-D-glucopyranose (2).

HS-142-1 is an ANP antagonist and is a mixture of β-1→6 oligoglucoside (DP: 10-30) partially acetylated with caproyl groups (5-15 in number).

In a particularly preferred embodiment of the above invention the adenosine receptor antagonist is 1,3-dipropyl-8-(3-noradamantyl)xanthine.

The ANP receptor antagonist is administered in a concentration which increases the effect of the natriuretic peptide, more particularly ANP in the following manner. Through the increased binding of ligands (without corresponding activation) on the ANP receptor, the receptor is blocked for its natural ligand. If treated with a limited concentration of antagonists (max. 95%, preferably max. 90% of the receptor are bonded, most preferred 80%) the new synthesis of the receptors induced by the occupied receptors results in an overall higher number of free receptors than before the treatment. The increase in receptors then results in an upward regulation of the natriuretic effect of the endogenous hormone as the balance of free ligand to bonded ligand is shifted in favour of the bonded ligand which generates the biological effect.

This effect occurs if at least 50%, preferably 70%, most preferably 80% but not more than 95% of the receptors are occupied by antagonists. In the case of HS-142-1 this usually corresponds to a dose of 0.01 mg to 0.1 mg HS 142-1/kg bodyweight, preferably 0.05 mg/kg.

It is known to a person skilled in the art that concentration ranges for antagonists, including ANP receptor antagonists, depend on the affinity of the antagonists. On the basis of the determined occupancy of the receptor and the affinity of the receptor, a person skilled in the art can determine the relevant dose of the relevant antagonist. (This determination can also be guided by the plasma concentration of pro-ANP, and for example at >74 pmol/l (plasma concentration) a range of 1× Pro-ANP 100× Pro-ANP can be defined, i.e. at 4 l Plasma and a Pro-ANP value of 100 pmol/l a range of 400 pmol-40 nmol antagonists/administration.)

The desired effect of the adenosine receptor antagonists occurs when at least 50%, preferably 70%, most preferably 80% but no more than 95% of the receptors are occupied by antagonists. Normally in the case of the preferred 1,3-dipropyl-8-(3-noradamantyl)xanthine this corresponds to a dose of 0.025 to 1 mg/kg bodyweight, preferably 0.5 mg/kg.

The above drugs are particularly useful if the clinically manifest neurodegenerative disorder is of Alzheimer's dementia type.

In a preferred embodiment the active substance is thus an ANP receptor antagonist and is administered at a dose at which at least 50%, preferably 70%, most preferably 80% but no more than 95% of the ANP receptors are occupied.

In another preferred embodiment the active substance is an adenosine receptor antagonist and is administered at a dose at which at least 50%, preferably 70%, most preferably 80% but no more than 95% of the adenosine receptors are occupied.

In a preferred embodiment these drugs are used to treat a particular patient group, whereby the group of patients is selected, who on the basis of an increase in circulation-relevant peptide biomarkers measured in their circulation, can be identified as patients at increased risk of developing a clinically manifest form of Alzheimer's dementia. An in-vitro method is also described in the following and is used for the selection of the patient group which is particularly identified as patients at increased of developing clinically manifest Alzheimer's dementia. In these patients treatment with the above ANP antagonists in accordance with the invention is especially recommended.

In a preferred embodiment of the invention the treatment is a preventive treatment.

Other ANP receptor antagonists can be identified by way of a screening method and also form the subject matter of the present invention. The subject matter of the invention is therefore a screening method for identifying antagonists of the receptors for the atrial natriuretic peptide comprising the following stages:
carrying out a competitive receptor assay to identify potential antagonists,
testing the ability of the potential antagonists to block ANP-induced cGMP formation and
identifying the antagonists of the receptors for the atrial natriuretic peptide.

Potential ANP receptor antagonist candidates can for example be found on the basis of a radio-receptor assay or a radio-immune assay in which candidates are identified which suppress the radioactively marked ANP. Such assays are known to a person skilled in the art and are described in the literature, e.g. in Capper et al. (1990) Clin. Chem. 36/4, 656.

Potential candidates are then tested for their capacity to block ANP-induced cGMP-formation. Appropriate assays are known to a person skilled in the art and can be obtained, for example, from the company CisBio international (http://www.htrf.com/products/gpcr/cgmp/).

In connection with the present invention, an ANP receptor antagonist is a compound which in the radio-receptor assay described below has an IC₅₀ value of under 10 nmol/l, preferably under 10 pmol/l and in an ANP-induced cGMP blockade assay has an IC₅₀ value under 10 nmol/l, preferably under 10 pmol/l.

The IC₅₀ value is the concentration of a (potential) antagonist (inhibitor) at which half maximal displacement of the ligand (ANP) from the ANP receptor or half maximal blocking of the ANP-induced cGMP formation is observed.

The radio-receptor assay is based on the displacement of a radioactively-marked ANP derivative from the binding sites of the ANP receptor by a (potential) antagonist and in connection with the present invention is carried out as follows:
1. Provision of the reconstituted membrane-bonded ANP receptor (guanylate cyclase A or B) from animal or human tissue (preferably renal cell preparations) or suitable cell lines with a natural or recombinant ANP receptor in a cell count of 100-100,000 cells/analysis in a 50 mM PBS buffer solution, pH 7.5.
2. Addition of 1 fM to 100 nM of the (potential) antagonist 2000-100,000 cpm radioactively-marked ANP 1-28, radio-iodised on Tyr 28, ca. 2200 Ci/mmol (radio-iodised by means of the chloramine T method known to a person skilled in the art) so that the ratio ANP/ANP receptor is between 100:1 and 1:100.
3. Incubation for 90 min at room temperature.
4. Centrifuging (30 min, 4° C., 2000 g) to separate receptors and supernatant
5. Determination of the concentration of free (supernatant) and receptor-bonded (pellet) radioactively-marked ANP peptide by detection of the radioactivity with a gamma meter.
6. The IC₅₀ value is determined from the concentration of receptor-bonded radioactively-marked ANP peptide as a function of the concentration of the added antagonist.

The HTRF® (Homogeneous Time Resolved Fluorescence) cGMP blockade assay (CisBio international, Bagnols-sur-Cèze, France) is based on the inhibition of ANP-induced cGMP formation by a (potential) antagonist. Use is made of the fact that the membrane-bonded guanylate cyclase (type 1) is activated by ANP. In the cGMP blockade assay the formation of cGMP through the present ANP receptor is determined in the presence of various concentrations of the (potential) antagonist. In a competitive immune assay the quantity of cGMP formed in each case compared with a fluorescence-marked cGMP of known concentration is then determined by means of an also fluorescence-marked anti-cGMP antibody.

In connection with the present invention the HTRF® cGMP assay is carried out as follows:
1. Provision of the membrane-bonded ANP receptor (guanylate cyclase) from animal or human tissue (preferable renal cell preparations) or suitable cell lines with a natural or recombinant ANP receptor in a cell count of 100-100,000 cells/analysis. In 50 mM PBS, 0.2% BSA, pH 7.2.
2. Addition of 1 fM to 100 nM of the (potential) antagonist and 5-100,000 pM ANP 1-28, human.
3. Incubation for 30 mins at 37° C.
4. Determination of the formed cGMP concentration by means of adding cGMP-d2 in lysis buffer and anti-cGMP cryptate (anti-cGMP antibodies in lysis buffer) in a competitive immuno assay. The cGMP concentration is proportional to the ratio of the fluorescence intensity at 665/620 nm). From the dependence of the formed cGMP on the relevant antagonist concentration the IC₅₀ value is determined.

Methods of diagnosing (in a broad definition of this term) neurodegenerative diseases through determination of circulation-relevant (vasoactive) peptides in, for example, serum or plasma samples of relevant patients are described in earlier applications by the applicant, more particularly in DE 10 2006 023 175 A1 and WO 2007/131 775 A1, where a statistically relevant correlation between the severity of a neurodegenerative disease and the measurable concentrations of natriuretic peptides in the patient samples is demonstrated, as well as in DE 10 2006 027 818 A1 and WO 2007/144 194 A1, where it is described that an even better, statistically more relevant correlation can be achieved if more than one vasoactive peptide is considered at the same time, more particularly in the form of a combination of concentrations of vasodilating peptides on the one hand, and vasoconstrictive peptides in the patient sample on the other hand.

The measurement described in said older applications took place using samples (more particularly plasma samples) of patients who had already been diagnosed with “developed neurodegenerative disease”, or who were at least suspected to be suffering from an early form of such a disease.

The measurements were not evaluated for prognostic purposes, as the further medical development of the individual patients whose samples were measured was not followed-up over longer periods and was not related to the previously measured biomarker concentrations.

However, said earlier applications contain detailed discussions of the accepted clinical definitions of different types of neurodegenerative diseases and the problem of their correct diagnosis. As the present application is at least partly based on the examination findings in the two cited applications, an interested person skilled in the art will, in addition to the following statements, be expressly referred to the entire contents of the two older applications.

As has been stated, neurodegenerative diseases (dementia diseases) generally tend to be slowly developing chronic diseases with a non-infectious aetiology. The long observation periods required for making a reliable diagnosis constitute a considerable problem, both in terms of the diagnosis and prognosis as such, but also with regard to researching said diseases and the development of effective prevention and treatment strategies.

Generally designated as dementia diseases (dementia) are conditions which have, as a common feature, the loss of acquired intellectual capacities, above all memory and normal personality level as a result of damage to the brain. If dementia symptoms do not occur at advanced age but in middle age, this is known as presenile dementia.

On the basis of typical symptoms and postmortally determined pathological brain abnormalities, dementia is made up of various diseases or groups of diseases:

Of these Alzheimer's dementia (Alzheimer's disease) is the most common neurodegenerative dementia disease. It constitutes ⅔ of all cases of dementia and is in practice the most important area of application for the present invention. AD is characterised by three important features, which however can only be determined with certainty post mortem: the formation of amyloid plaques and neurofibrillar bundles as well as the loss of nerve cells (cf. also the introduction to the descriptions of the above earlier applications).

Other dementia diseases which are of lesser importance within the framework of this invention and are therefore not discussed in detail here are the so-called vascular dementias (VAD) in which dementia occurs through blood supply disorders in the brain. There are different types of VAD, whereby multi-infarction dementia (MID) and sub-cortical VAD (also known as Binswanger's disease) are the most common forms. Also, for the sake of completeness, dementia with Lewy bodies (DBL) and frontotemporal dementia (FTD), also known as Pick's disease should be mentioned.

The brain changes typical of the various dementia diseases cannot of course be directly determined in living patients, and technical examinations of the brain function with, for example, X-rays or magnetic resonance imaging, are laborious and expensive. Even today dementia diseases are therefore underdiagnosed or incorrectly diagnosed, and prognosis and treatment possibilities are correspondingly poor. The work of the applicant is therefore aimed at improving the diagnosis and prognosis possibilities for neurodegenerative diseases, in particular the diagnosis of AD, through the development of relatively simple to use measuring methods as part of which easily measurable biomarkers are identified in samples from the patients' blood circulation.

The promising biomarkers in accordance with the invention include various circulation-relevant peptide biomarkers, from which as part of the method of the present invention biomarkers are determined in the form of peptides with a vasodilator action, more particularly natriuretic peptides such as ANP, BNP and/or CNP and/or the peptide adrenomedullin (ADM).

As an example, the determination of the concentrations of sodium diuretic peptides is shown through determining the concentration of ANP. ANP is preferably determined as a concentration of a pro-ANP fragment with the aid of an assay by the applicant which identifies a middle section of the proANP (MR-proANP). An assay of this type is described in EP 1 562 984 B1 and in: Nils G. Morgenthaler et al., Immunoluminometric Assay for the Midregion of Pro-Atrial Natriuretic Peptide in Human Plasma, Clinical Chemistry 50:1, 2004, 234-236.

The concentration of adrenomedullin (ADM) is preferably determined as the concentration of a mid-regional proADM fragment (MR-proADM) that comprises the amino acids 45-92 of the pre-proadrenomedullins. A suitable assay is described in EP 1 488 209 B1 and WO 2004/090546 and in Nils G. Morgenthaler et al., Measurement of Midregional Proadrenomedullin in Plasma with an Immunoluminometric Assay, Clinical Chemistry 51:10, 2005, 1823-1829.

If a term such as “concentration of a peptide biomarker” or a similar term is used in this application, this does not only mean the stationary concentration of the actual vasoactive peptide measurable in the biological sample in the sense of a limiting equalization.

The most important pathophysiologically released actual vasoactive peptides discussed in the context of the present invention are only present in freely or unrestrictedly measurable form in biological fluids in small quantities. Considerable portions of the pathophysiologically released actual vasoactive peptides are rapidly removed from the biological fluid and/or degraded through binding to receptors and other membrane or vascular structures.

The measurement of inactive co-peptides formed from the same precursor pro-peptides, as preferably carried out in the context of this invention using the applicant's said assays, reflects, in contrast to the measurable momentary concentration in a biological fluid, the release of the vasoactive peptides in the sense of “active concentrations” over a longer period and allows the indirect co-recording also of bonded and or rapidly degraded portions of the originally released vasoactive peptide. In conjunction with the greater stability of such co-peptides, this leads to higher measurable absolute concentration values for the analyte of interest in the biological fluid, e.g. in serum or plasma.

In the case of diseases which tend to have chronic course without sudden deteriorations or improvements in the patient's condition, as is generally the case in dementia diseases, there is a considerable likelihood that with regard to the various disease-relevant analytes an only slightly variable, only slowly changing stationary overall condition will develop. The concentrations of an active analyte of a peptide biomarker type, in this case a vasoactive peptide, stationary measurable in the biological fluid of the patient, may therefore be essentially proportional to the pathophysiologically released quantities of the same analyte over longer periods, as can be measured in the form of the physiologically inactive co-peptide. This means that the deviations from the control values of healthy persons determined in the preferred determination of inactive co-peptides in accordance with the present invention, as well as the disease-typical course of these deviations should also be reflected in the generally lower stationary concentrations of the active peptides, so that the specific selection of the measured “analyte concentrations” should have no decisive qualitative influence on diagnostic evaluation.

As part of the present invention, for measuring purposes a collection of samples of patient plasma could be used which included the plasma samples of patients obtained at the time of being diagnosed with “mild cognitive disorders” (t=0) (“MCD patients”). The subsequent development of the state of health of the individual patients was then documented for a period of 6 years.

It was found that the concentrations of the examined biomarkers in the plasma of MCD patients was on average increased compared with control persons without cognitive disorders, and that within the range of the found concentrations a cut-off concentration value could be defined which allowed MCD patients at low risk of subsequent conversion to AD to be distinguished from MCD patients at considerable risk of subsequent conversion to AD. The distinction between the patient groups was improved further through the simultaneous consideration of two biomarkers.

As the inventor, as will be shown in the experimental section, also examined the results, based on the documented medical histories, to see whether patients in whom no or clearly delayed subsequent conversion in the sense of the development of a clinically manifest neurodegenerative disease (Alzheimer's dementia) occurred, had already for other reasons undergone treatment with certain medications or types of medication, he was able to show that evidently treatment with cardiovascular agents, more particularly antihypertensive agents, had had a positive influence on the prognosis in the form of avoidance or delay of “conversion to AD”.

This showed that for patients with mild cognitive disorders not only is risk stratification possible at the time of diagnosis, but that patients with a determined increased risk of conversion to AD can be recommended a rational new treatment, which does not currently form part of the treatment repertoire of key specialists, namely treatment with cardiovascular agents, more particularly antihypertensive agents, or treatment with ANP receptor antagonists or adenosine receptor antagonists.

Cardiovascular agents and antihypertensive agents in accordance with the above are known agents which have undergone extensive clinical testing. The antihypertensive agents which are routinely used therapeutically and/or as prophylaxis today essentially represent four different types of drug, namely agents in the group of inhibitors of Angiotensin Converting Enzyme (ACE inhibitors), angiotensin II receptor antagonists (“sartans”), beta blockers and diuretics used to lower blood pressure.

Further details of the currently used types of these agents and the widely prescribed representatives of said active substance classes can be found in the very extensive specialist literature concerned with reducing the blood pressure of hypertensive patients.

Listed solely as examples of ACE inhibitors are the pharmaceuticals Captopril, Enalapril, Lisinopril and Ramipril, as representatives of angiotensin II receptor subtype 1 antagonists (AT1 antagonist; sartan) the pharmaceuticals Losartan, Valsartan, Candesartan, Irbesartan, Telmisartan, Eprosartan and Olmesartan, and as diuretics, in particular so-called thiazide diuretics such as hydrochlorothiazide and the thiazide analogues such as Clorthalidon, Clopramide, Indepramide, Metolazon, Xipramide and Mefruside.

The following table 1 briefly summaries the basic information about the various types of agents and their action mechanisms:

TABLE 1

| Agent | Effect |
|---|---|
| | They block the action of the endogenous protein |
| | ACE, which is responsible for producing the |
| | hormone angiotensin II (AT-II). AT II narrows |
| | the blood vessels and keeps the blood pressure |
| | high. By reducing the AT II concentration in |
| | the body the blood vessels remain dilated and |
| | the blood pressure decreases. |
| ACE inhibitors | The heart has to work against less resistance |
| | and is relieved of strain. They can also delay |
| | pathological conversion of the heart muscle. |
| | ACE inhibitors improve the prognosis of cardiac |
| | insufficiency and are well tolerated. A tickly |
| | cough often occurs, but this is harmless. |
| Angiotensin II | These are also known as “sartans” and compete |
| receptor | with angiotensin II for the binding sites of |
| antagonist | the corresponding receptors. They are tolerated |
| | better by various patients and exhibit a better |
| | effect in various patients than ACE inhibitors |
| | and are used if a patient cannot tolerate ACE |
| | inhibitors, or if they do not provide the |
| | desired effect. |
| Beta blockers | They reduce the effect of stress hormones |
| | (catecholamines) on the heart. In cardiac |
| | insufficiency increased stress hormones are |
| | produced and can lead to heart rhythm |
| | disorders. Beta blockers improve the |
| | prognosis as they prevent life-threatening |
| | cardiac rhythm disorders. |
| Diuretics | Special urine-promoting agents (aldosterone |
| | antagonists) improve the prognosis in |
| | advanced stages. The precise mode of action |
| | is still unclear. |

In another aspect, the present invention relates to an ANP receptor antagonist for use as a medicament.

Preferably the ANP receptor antagonist is a glucose-caproic acid polymer.

In another aspect, the present invention relates to an adenosine receptor antagonist for use as a Medicament.

Preferably the adenosine receptor antagonist is 1,3-dipropyl-8-(3-noradamantyl)xanthine (“rolofylline”).

In medicine the term “risk stratification” (or simply “stratification”) denotes assessing the risk of a disease progressing and resulting in increasingly serious complications or to a predefined endpoint (e.g. death due to the disease) in a patient or in a subgroup of a test population of patients. For risk stratification, risk factors or diagnostically relevant parameters are recorded which are known to be associated with the progression of the disease and/or the occurrence of complications. With the aid of various statistical biometric evaluation methods in the form of tables, algorithms or computer programs working with these, the individual patient risk or patient subgroup risk is determined on the basis of the recorded data.

Risk stratification can also be considered as a prognosis tool with which a prognosis, which is taken to mean a certain probability, is derived from test criteria which in previous studies have proven to be relevant and/or valid for the issue in question, e.g. in order to initiate any countermeasures in good time if there are rational preventive therapies for the developing disorders, or to prevent those patients having to undergo stressful and/or expensive treatment and prevention measures who are most unlikely to need them.

A known mathematical method of risk stratification which is adapted to special test conditions is the so-called Kaplan-Meier method (also known as “Kaplan-Meier estimator”), which, for example, is used for survival analysis if not all the test subjects included in the test are available for the entire duration of the test, e.g. because they are only included in the patient population at a later date or they leave it, e.g. due to death not as result of the examined risk, or because they exhibit no clear findings over the test duration.

Said evaluation method which is described in more detail in textbooks on medical biometry, for example, and/or for which under the above designations there are many explanations on the internet, and for the application of which commercial computer programs are available, is applied for the purposes of the evaluation of the measurement, described in the following experimental section, of circulation-relevant peptide markers in the plasma of patients with an initial diagnosis of “mild cognitive disorder”. When using this method so-called “Kaplan-Meier plots” are obtained as shown in most of the figures of the present application.

In the following experimental section, the measurements forming the basis of the present invention and the corresponding evaluations of the measuring results, are explained in more detail with reference to several figures and tables.

Reference is made to ten figures (diagrams), the content of which is explained in more detail below:

FIG. 1 shows a so-called Kaplan-Meier plot in which the development of the state of health in the sense of conversion to Alzheimer's, i.e. development of an AD, of the measuring population of 131 patients with the initial diagnosis “mild cognitive disorders” is shown on the basis of the purely clinical diagnosis over a period of more than six years. During the monitored period 57 of the 131 patients developed clinically manifest Alzheimer's dementia (AD).

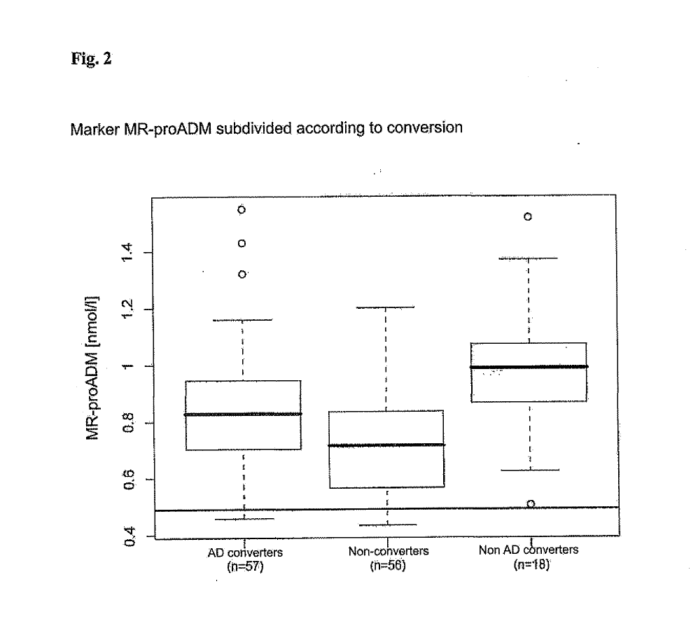

FIG. 2 shows the concentrations of the peptide biomarker MR-proADM measured in the plasma samples of the group of patients in FIG. 1 at the time of diagnosis (t=0) separately for the subsequent converters to Alzheimer's (AD converters), the non-converters and converters to other “non-AD” forms of dementia.

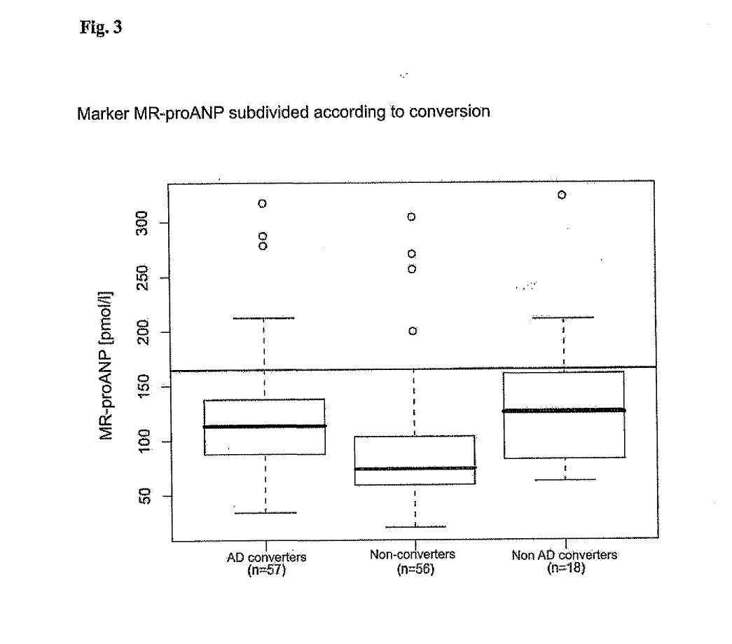

FIG. 3 shows the concentrations of the peptide biomarker MR-proANP measured in the plasma samples of the group of patients in FIG. 1 at the time of diagnosis (t=0) separately for the subsequent converters to Alzheimer's (AD converters), the non-converters and converters to other “non-AD” forms of dementia.

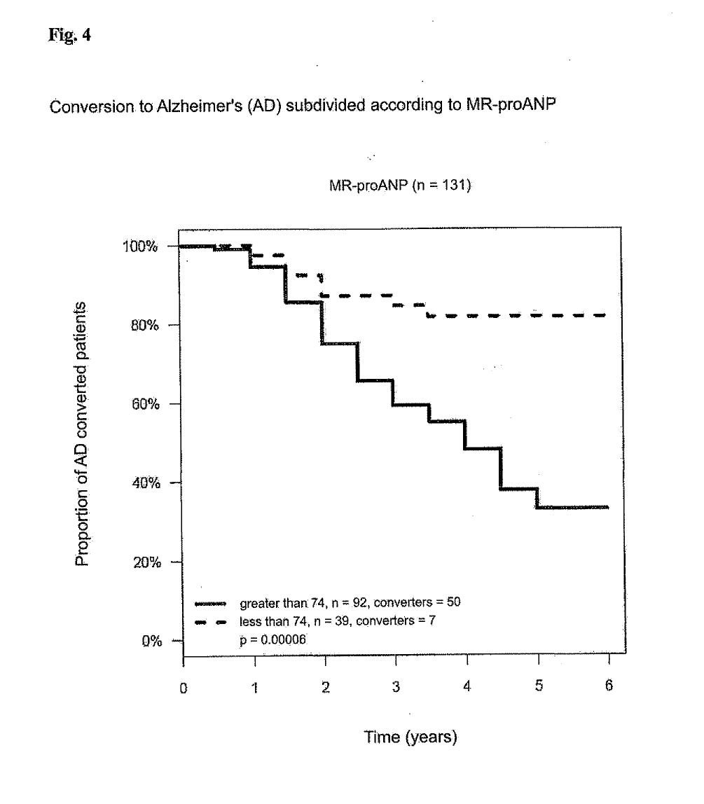

FIG. 4 shows a Kaplan-Meier plot for conversion to AD for two groups of patients, on the one hand with a measured MR-proANP concentration above the cut-off value of 74 pmol/l (greater than 74) and on the other with a concentration below said cut-off value (less than 74).

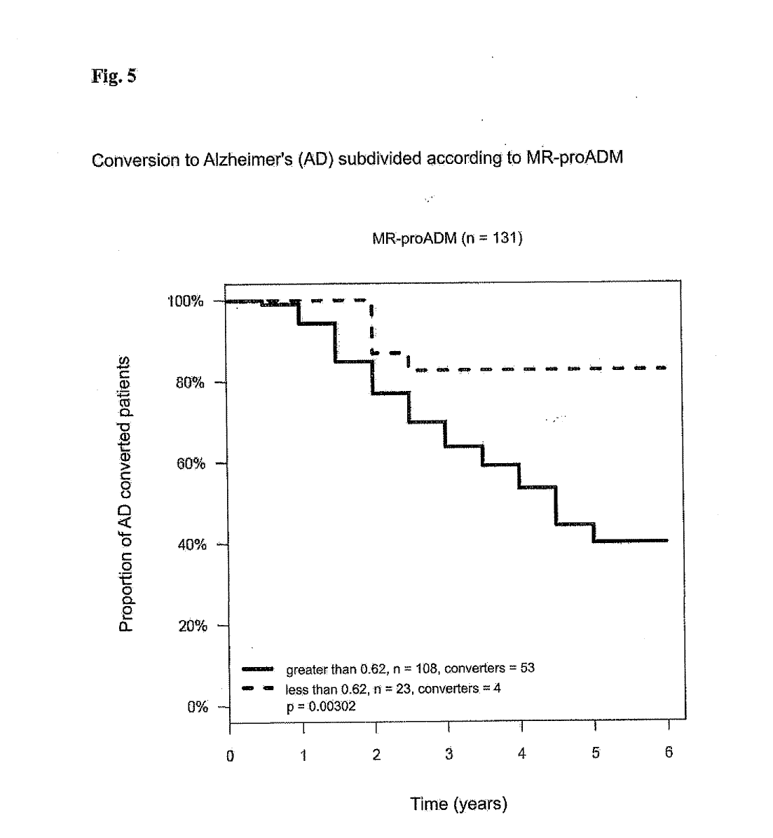

FIG. 5 shows a Kaplan-Meier plot for conversion to AD for two groups of patients, on the one hand with a measured MR-proADM concentration above the cut-off value of 0.62 nmol/l (greater than 0.62) and on the other with a concentration below said cut-off value (less than 0.62).

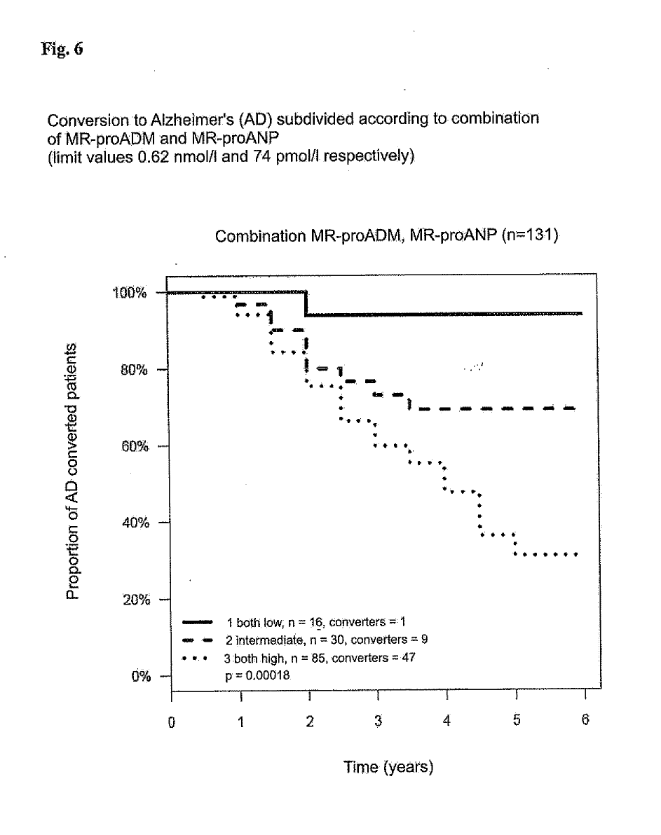

FIG. 6 shows a Kaplan-Meier plot for conversion to AD in patients wherein the plasma concentrations for both peptide biomarkers MR-proANP and pro-ADM were taken into consideration at the same time, divided into patient groups in whom (a) the concentration of both biomarkers was below the cut-off values set out in FIGS. 4 and 5 (both low), (b) only one of the concentrations was below one of the cut-off values set out in FIGS. 4 and 5 (intermediate), and (c) the concentrations of both biomarkers were above the cut-off values set out in FIGS. 4 and 5 (both high).

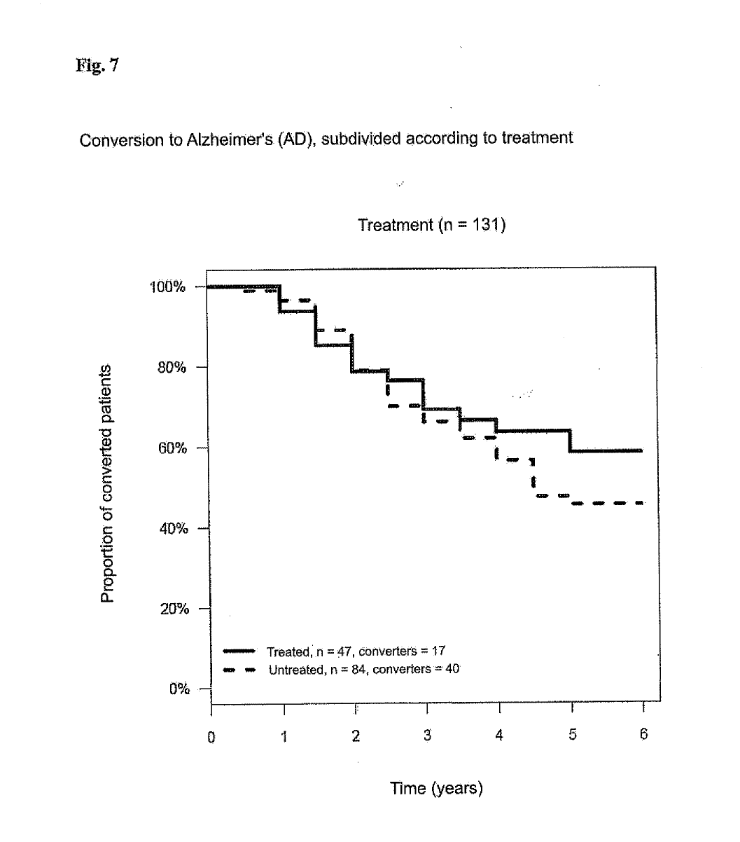

FIG. 7 shows the clinically diagnosed conversion to AD of the 131 MCD patients divided into patients who according to their medical records had been treated with cardiovascular agents before diagnosis (treated), and patients for whom no such previous treatment was documented. On the basis of such an evaluation no significant difference can be determined between these patient groups.

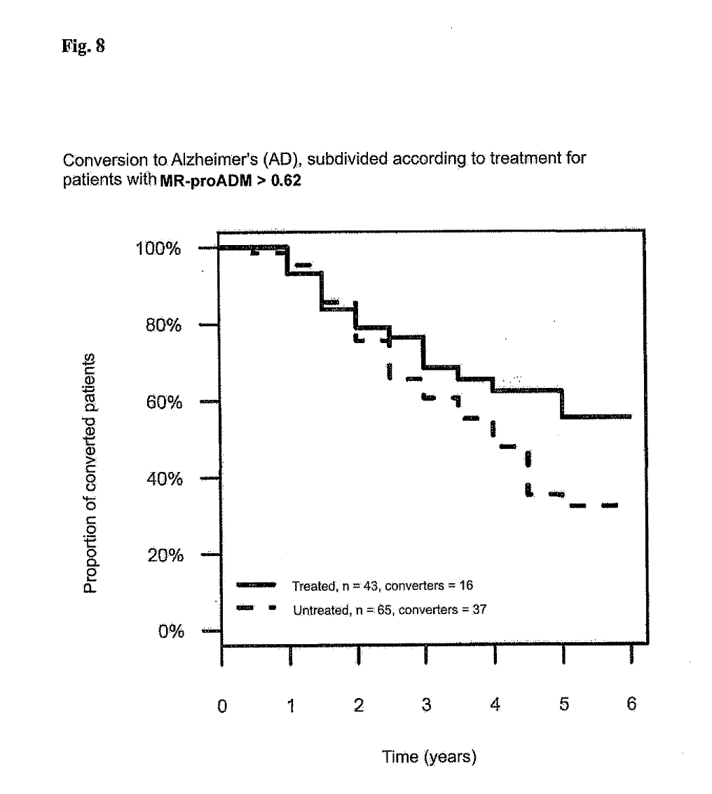

FIG. 8 shows the clinically diagnosed conversion to AD of a subgroup of 108 patients of the 131 original MCD patients in whom an MR-proADM value above the cut-off of 0.62 nmol/l was measured, divided into patients who according to their medical records had been treated with cardiovascular agents before diagnosis (treated), and patients for whom no such previous treatment is documented (untreated).

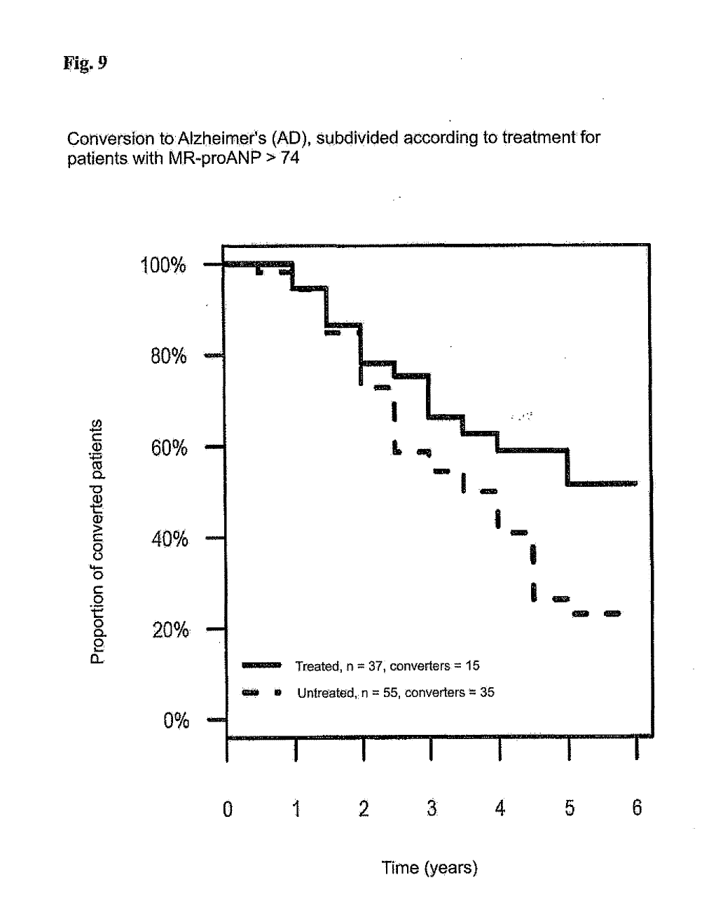

FIG. 9 shows the clinically diagnosed conversion to AD of a subgroup of 92 patients of the 131 original MCD patients in whom an MR-proANP value above the cut-off of 74 pmol/l was measured, divided into patients who according to their medical records had been treated with cardiovascular agents before diagnosis (treated), and patients for whom no such previous treatment is documented (untreated).

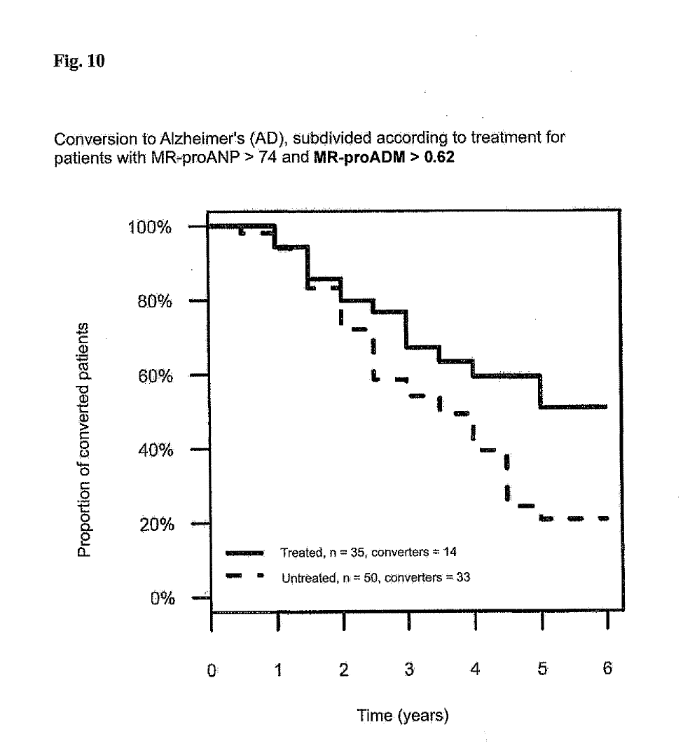

FIG. 10 shows the clinically diagnosed conversion to AD of a subgroup of the 85 patients of the 131 original MCD patients in whom both an MR-proANP-value above the cut-off of 74 pmol/l as well as an MR-proADM value above the cut-off of 0.62 nmol/l was measured, divided into patients who according to their medical records had been treated with cardiovascular agents before diagnosis (treated), and patients for whom no such previous treatment is documented (untreated).

### EXPERIMENTAL SECTION

### Patient Population

From 131 patients diagnosed with mild cognitive disorders (MCD or MCI) with a median age of 71.5 years (n=131) EDTA blood samples were obtained at the time of first diagnosis (t=0) from which EDTA plasma samples were produced as study material for biomarker measurements.

Over the following 6 years the patients were regularly examined at 6-monthly intervals. Each time it was recorded whether conversion to a clinically manifest neurodegenerative disease (Alzheimer's dementia, vascular dementia) had developed.

Alzheimer's dementia was diagnosed in accordance with the NINCDS-ADRDA criteria (McKhann et al., Neurology 1984; 34: 939-944). MCI was diagnosed according to the criteria by Petersen (Arch Neurol 1999; 56: 303-308).

During the observation period out of the 131 patients 75 patients (converters) developed a documented neurodegeneration (n=60 Alzheimer's dementia, n=15 vascular dementia), 56 patients (non-converters) remained in MCD status.

FIG. 1 shows said findings for the entire patient population in the form of a Kaplan-Meier plot based solely on the clinical diagnosis.

Table 2 shows the number of MCD patients (patients at risk of developing a form of dementia) and their clinical development over the observation period expressed as the number of patients exhibiting conversion to Alzheimer's (AD converters), those exhibiting conversion to another form of dementia (non-AD converters) and those who exhibited no conversion (non-converters) as well as the percentage of AD converters related to the number of patients in the study.

TABLE 2
Conversion to Alzheimer's (AD)

| | Inclusion | 12 months | 24 months | 36 months | 48 months | 60 months |
|---|---|---|---|---|---|---|
| Patients in | 131 | 131 | 131 | 131 | 125 | 106 |
| the study | | | | | | |
| AD | 0 | 6 | 27 | 41 | 50 | 57 |
| converters | | | | | | |
| Non-AD | 0 | 3 | 8 | 17 | 17 | 18 |
| converters | | | | | | |
| Non- | 131 | 122 | 96 | 73 | 58 | 31 |
| converters | | | | | | |
| Proportion of | 0% | 4.6% | 20.6% | 31.3% | 40.0% | 53.8% |
| AD converters* | | | | | | |

*Calculated in relation to the patients in the study

### Biomarker Measurements/Assay Description and Evaluation

In the EDTA plasma samples obtained at the time of diagnosis the concentrations of the peptide biomarkers MR-proANP and MR-proADM were measured using the applicant's assays.

Measurement of MR-proANP in the plasma was essentially carried out with an immunoluminometric sandwich assay as described in the experimental section of the aforementioned WO 2004/046181.

Measurement of MR-proADM in the plasma was essentially carried out with an immunoluminometric sandwich assay as described in the experimental section of the aforementioned WO 2004/090546.

As shown in the cited earlier patent applications, for healthy persons (60 symptom-free control persons exhibiting no symptoms of cognitive disorders and not suffering from any detectable illness, for which it is known that increased levels of the relevant biomarker analyte can be measured) medians for the measured concentrations are determined as follows:

MR-proANP=62.3 pmol/l
MR-proADM=0.60 nmol/l.

### Evaluations

The concentrations of the peptide biomarkers measured in the plasma of the 131 MCD patients obtained at the time of diagnosis are shown in FIG. 2 (for MR-proADM) and in FIG. 3 (for MR-proANP) respectively, separately for subsequent converters to AD, converters to other forms of dementia (vascular dementia) and non-converters.

It was found that for both biomarkers even at the time of initial diagnosis the medians of the groups of the various converters and the non-converters differ significantly.

As shown in summarized form in table 3 below, the medians of the concentration values for both biomarkers measured for the subsequent converters are significantly increased.

TABLE 3

| | MR-proADM | MR-proANP |
|---|---|---|
| Non-converters | 0.71 nmol/l | 73 pmol/l |
| Non AD converters | 0.99 nmol/l | 125 pmol/l |
| AD converters | 0.83 nmol/l | 113 pmol/l |

Determination of the optimum cut-off value for each biomarker for further evaluation took place on the basis of so-called ROC plots (not shown). It was found that the cut-off values are sometimes only relatively slightly higher than the medians determined for the control persons.

FIGS. 4 and 5 show the Kaplan-Meier plots obtained when applying the above cut-off values to the population of MCD patients, divided into patient groups with concentration values of the indicated peptide marker above the relevant cut-off (greater) and below it (smaller).

As can be seen in FIGS. 4 and 5 in the case of measuring MR-proANP and MR-proADM concentrations in the plasma of MCD patients which at the time of initial diagnosis are above the cut-off values determined for said assays, there is a considerably greater probability of subsequent conversion for these patients than in the case of lower values.

The risk of developing Alzheimer's dementia-type neurodegenerations is therefore associated with the increase in the selected blood markers. Of the patients for whom the MR-proADM concentrations in the plasma were measured below the cut-off of 0.62 nmol/l MR-proADM only 17.4% developed an AD disease whereas 60.1% of patients with values above said cut-off converted to AD. This risk of conversion in the case of concentrations above the cut-off is therefore around 3.45 higher.

Similarly 67.1% of patients with values above the cut-off of 74 pmol/l for MR-proANP converted, but only 18.2% of those patients for whom MR-proANP concentrations below this were measured; the risk for patients with MR-proANP concentration values above the cut-off thus increases by a factor of around 3.7.

By combining the biomarkers the risk prognoses can be improved even further: by way of example, in the evaluation of the above measuring results 3 patient groups were therefore created: a) patients with both markers below the selected cut-off values, b) patients with one of the markers above the relevant cut-off and c) patients with both markers above the relevant cut-off values. FIG. 6 shows the Kaplan-Meier plot obtained with such an evaluation.

It can be seen from FIG. 6 that if both biomarkers are jointly taken into account patients in whom both biomarkers were found to be below the relevant cut-off points at the time of diagnosis (t=0) are very likely to belong to the group of non-converters, whereas when both biomarkers are found to be above the corresponding cut-off values the probability conversion to AD within 5 years is very high for such patients.

The relative risk of developing a manifest neurodegeneration of AD type between the group “both markers below cut-off” (6.8% conversion) and “both markers above cut-off” (69% conversion) increases to around 11.

Evaluation Taking into Account Previous Treatments of the Patients with Cardiovascular Agents

The measured biomarkers ADM and ANP, the release of which into the circulation was indirectly determined by way of the MR-proANP and MR-proADM fragments respectively, are known as biomarkers for cardiac insufficiency. There is therefore a strong association between elevations in biomarkers for cardiac insufficiency and the development of neurodegenerative diseases, more particularly Alzheimer's dementia. However, the correlation is shown at a surprisingly low level of biomarker concentrations which lie within the distribution of “normal concentrations” determined for the relevant markers. Slight increases in the observed markers therefore tend to be involved. Working back from this it was concluded that in patients who subsequently develop a neurodegeneration, more particularly of AD type, an only slight, but continuous deterioration of circulatory efficiency can be observed.

On the basis of this the inventor developed the hypothesis that stabilisation of the circulation of MCD patients with slightly elevated circulation-relevant peptide biomarker concentrations could be effective in preventing or delaying conversion.

However, if, on the basis of the above considerations, the development of all 131 MCD patients is examined in terms of the development of a form of clinically manifest Alzheimer's dementia (AD) taking into account the aspect of “previously treated with cardiovascular agents before diagnosis” and “not previously treated with cardiovascular agents before diagnosis” no significant differences between the two groups of patient can be determined. In this context, reference is made to FIG. 7.

However, the situation changes if not all the MCD patients are taken into account but only those for whom in the above biomarker measurements plasma concentrations of the circulation-relevant biomarkers above the relevant cut-off values have been determined.

If on the basis of the documented medical history of the MCD patients in the studied patient population it is examined how many of the patients, who at the time of being diagnosed with “MCD” had plasma concentration values of one or both of the studied peptide biomarkers MR-proANP and/or MR-proADM above the selected cut-off values, had for other reasons already been treated with at least one of the drugs listed in table 1 above, and the extent to which such treatment is reflected in the observed AD conversion rates in said patient groups, clear correlations are seen in that patients in whom one or both of the measured circulation-relevant biomarkers was/were elevated at the time of being diagnosed with MCD and who had been previously treated with products in the cited class of cardiovascular agents, exhibited significantly less conversion to AD than corresponding patients who had not received such previous treatment.

The following tables 4, 5 and 6 summarise the results of such an evaluation of the patient data, respectively taking into consideration only patients in whom MR-proANP was found to be elevated (table 4), for whom MR-proADM was found to be elevated (table 5), and for patients in whom both biomarkers were found to be elevated.

TABLE 4
Conversion to Alzheimer's (AD), MR-proANP >74 pmol/l

| | Treated | Untreated |
|---|---|---|
| Total | 37 | 55 |
| AD converters | 15 | 35 |
| Proportion of AD | 41% | 64% |
| converters | | |

Conversion to AD during treatment in patients with MR-proANP>74, p=0.035

Decrease in conversion to AD with treatment: 35.9%

TABLE 5
Conversion to Alzheimer's (AD), MR-proADM >0.62 nmol/l

| | Treated | Untreated |
|---|---|---|
| Total | 43 | 65 |
| AD converters | 16 | 37 |
| Proportion of AD | 37% | 43% |
| converters | | |

Conversion to AD on treatment in patients with MR-proADM>0.62, p=0.051

Decrease in conversion to AD on treatment: 14.0%

TABLE 6
Conversion to Alzheimer's (AD), MR-proANP >74
pmol/l and concurrent MR-proADM >0.62 nmol/l

| | Treated | Untreated |
|---|---|---|
| Total | 35 | 50 |
| AD converters | 14 | 33 |
| Proportion of AD | 40% | 66% |
| converters | | |

Conversion to Alzheimer's (AD) with MR-proANP>74 and MR-proADM>0.62, p=0.026

Decrease in conversion to AD on treatment: 39.4%

The contents of tables 4, 5 and 6 are shown graphically in FIGS. 8, 9 and 10 respectively in the form of Kaplan-Meier plots.

From the tables and accompanying figures it can be seen that the conversion rate to AD of pharmacologically previously treated patients who at the time of diagnosis had plasma concentrations of the measured peptide biomarkers above the set cut-off values was very much lower, i.e. fewer clinically manifest AD diseases developed than in the case of comparable patients who, however, had not received previous pharmacological treatment.

From this considerable improvements in the prognosis of patients with MCD (or MCI) for whom (slightly) elevated circulation-relevant biomarkers were measured can be derived if such patients are treated in good time with one or more cardiovascular agents (antihypertensives, products to treat cardiac insufficiency).

Consequently in accordance with the present invention, for MCD patients in whom a previous in vitro analysis has found increased values of peptide biomarkers (ANP; ADM), i.e. who tend to have a poor prognosis, treatment with cardiovascular agents can be directly recommended as this can reduce the determined risk of conversion to AD.

The invention therefore also relates to a method for the prognosis and treatment of mild cognitive disorders and/or methods of identifying patients requiring treatment and for the preventive treatment of such patients with mild cognitive disorders.

1.-18. (canceled)

19. An in vitro method of identifying patients with mild cognitive disorders requiring treatment comprising
determining in a sample of a biological fluid from the circulation of patient diagnosed with a mild cognitive disorder at least one circulation-relevant peptide biomarker and
allocating an increased risk of developing a clinically manifest neurodegenerative disease to a concentration of the at least one circulation-relevant peptide biomarker exceeding a biomarker-specific threshold value (cut-off) set within the range of usual concentration values for this biomarker.

20. The in vitro method in accordance with claim 19 wherein for a patient with the determined higher risk a treatment is recommended with a drug comprising one or more active substances of cardiovascular agents selected from the group comprising: ANP receptor antagonists, adenosine receptor antagonists, ACE inhibitors, angiotensin II receptor antagonists, betablockers and antihypertensive diuretics and combinations thereof for the treatment of patients with mild cognitive disorders in order to delay or prevent the development of clinically manifest neurodegenerative disorders or to improve or maintain the status of the mild cognitive disorder.

21. The method in accordance with claim 20 wherein the ANP receptor antagonist is a glucose-caproic acid polymer.

22. The method in accordance with claim 20 wherein the adenosine receptor antagonist is 1,3-dipropyl-8-(3-noradamantyl)xanthine.

23. The method in accordance with claim 20 wherein the active substance is an ANP receptor antagonist and the antagonist is administered at a dose at which at least 50% of the ANP receptors are occupied.

24. The method in accordance with claim 20 wherein the active substance is an adenosine receptor antagonist and the antagonist is administered at a dose at which at least 50% of the adenosine receptors are occupied.

25. The method in accordance with claim 20 wherein the clinically manifest neurodegenerative disorder is of Alzheimer's dementia type.

26. The method in accordance with claim 20 wherein for treatment a group of patients is selected, who on the basis of an elevation of circulation-relevant peptide biomarkers measurable in their circulation can be identified as patients at increased risk of developing a clinically manifest form of Alzheimer's dementia.

27. The method in accordance with claim 19 characterised in that the sample is a serum or plasma sample.

28. The method in accordance with claim 19 characterised in that the circulation-relevant peptide biomarkers are selected from the natriuretic peptides ANP and/or BNP and/or adrenomedullin (ADM) or from fragments of their associated prohormones pro-ANP, pro-BNP or pro-ADM.

29. The method in accordance with claim 19 characterised in that a treatment is recommended with a cardiovascular agent from the group of antihypertensive agents selected from the group of “Angiotensin Converting Enzyme” inhibitors (ACE inhibitors), angiotensin II receptor antagonists (“sartanes”), beta blockers and diuretics used to lower blood pressure.

30. The method in accordance with claim 28, characterised in that determination is carried out with an immunodiagnostic determination method and that fragments of prohormone precursors of circulation-relevant peptides with a reduced or undetectable physiological effect vis-à-vis the actual circulation-relevant peptides are determined.

31. The method in accordance with claim 30 characterised in that the concentration of the mid-section of the pro-ANP fragment (MR-pro-ANP) containing the pro-ANP, and/or of a mid-regional pro-ADM fragment (MR-proADM) containing the amino acids 45-92 of the pre-proadrenomedullin is determined

32. A method for the stratification or identification of patients with mild cognitive disorders exhibiting an increased risk of conversion to a clinically manifest neurodegenerative disorder, comprising using circulation-relevant peptide biomarkers, more particularly natriuretic peptides ANP, BNP and/or adrenomedullin (ADM) or fragments thereof or fragments of their associated prohormones pro-ANP, pro-BNP or pro-ADM.
